(19) United States
(12) Patent Application Publication
(10) Pub. No.: US 2018/0050075 A1
(43) Pub. Date: Feb. 22, 2018

(54) THERAPEUTIC COMPOSITIONS AND USES THEREOF FOR ALLEVIATING PAIN SYSTEMS

(71) Applicants: Farah Kashefi, Vancouver (CA); Arash Mojtabavi Naeini, Vancouver (CA)

(72) Inventors: Farah Kashefi, Vancouver (CA); Arash Mojtabavi Naeini, Vancouver (CA)

(21) Appl. No.: 15/239,731

(22) Filed: Aug. 17, 2016

(51) Int. Cl. A61K 36/48; A61K 36/73; A61K 36/534; A61K 36/88; A61K 9/00

(52) U.S. Cl. CPC A61K 36/48; A61K 36/73; A61K 9/0014; A61K 36/88; A61K 36/534

(57) ABSTRACT

A therapeutic composition for skin use composed of essential oils derived from natural plant sources and at least one carrier oil. The compositions are administered topically to provide pain relief.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of bio-affecting and body treating compositions and more specifically relates to therapeutic compositions for skin use.

### 2. DESCRIPTION OF RELATED ART

Millions of individuals suffer from acute or chronic pain every year. In 2012, a study by health economists from Johns Hopkins University estimated the total cost of chronic pain in the United States to be well over 600 billion annually. The Johns Hopkins study highlights the staggering impact of acute and chronic pain on a nation's health-care system, work productivity, and health insurance costs. Beyond the measurable economic impacts, the human suffering associated with pain is incalculable.

Conventional treatments for pain include the prescribing of pharmacologic analgesics such as acetaminophen, NSAIDs such as Ibuprofen, cyclooxygenase 2 (COX-2) inhibitors, and synthetic opioids (narcotic painkillers) such as codeine, oxycodone, and the like. It is well known that these drugs are poorly tolerated by many individuals and carry with their use substantial risks, particularly in pregnant patients, elderly patients, and individuals with certain pre-existing conditions. As examples, acetaminophen can be toxic to patients with liver disease, long-term NSAID use has been associated with gastric disorders, and narcotic painkillers can produce a myriad of serious side effects including respiratory depression, nausea, and chemical addiction. For the reasons stated above, it is clear that alternatives to conventional drug therapies, and the risks associated with their use, would be of great benefit to many who suffer from acute or chronic pain.

Several attempts have been made to solve the above-mentioned problems using alternate bio-affecting and body treating compositions, such as those found in U.S. Pat. No. 7,368,135 to Anderson disclosing “a chemical formula for the composition of a liquid topical analgesic” that “comprises at least olive oil, castor oil, grapeseed oil, almond oil, apricot kernel oil, Vitamin E, various herbal essential oils, and a stabilizer.” Other related art includes U.S. Pub. No. 20120225143 to Soliz et al.; U.S. Pub. No. 20150110903 to Khan et al.; U.S. Pub. No. 20090022826 to Shrier et al.; U.S. Pub. No. 20120141575 to Kassner et al.; U.S. Pat. No. 6,280,751 to Fletcher et al.; U.S. Pat. No. 5,032,400 to Wiersum et al.; U.S. Pat. No. 6,582,736 to Quezada; U.S. Pat. No. 6,579,543 to McClung; U.S. Pat. No. 8,623,335 to Waddington; U.S. Pat. No. 8,017,162 to Shimoda et al.; U.S. Pat. No. 6,444,238 to Weise; U.S. Pat. No. 8,765,194 to Hutsell; Canadian Pat No. CA8017162 to Chaudhary et al.; Chinese Pat No. CN103006852 to Shuilian; and, Chinese Pat No. CN104258455 to Liang. This art is representative of bio-affecting and body treating compositions; however, none of the above references, taken either singly or in combination, is seen to describe the invention as claimed.

### BRIEF SUMMARY OF THE INVENTION

In view of the foregoing disadvantages inherent in the known bio-affecting and body treating composition art, the present invention provides novel therapeutic compositions for skin formulated from constituents derived from natural plant sources. Furthermore, preferred therapeutic compositions of the present system are formulated to contain no artificial colors or other synthetic chemicals. The general purpose of the present system, which will be described subsequently in greater detail, is to assist pain relief.

A therapeutic treatment system is disclosed herein, in a preferred embodiment, a composition for skin is provided that preferably includes: fenugreek essential oil, almond essential oil, mint essential oil, garlic essential oil, castor essential oil, and at least one carrier oil. Moreover, in such a preferred composition, the at least one carrier oil preferably includes fractionated coconut oil. Additionally, in such a preferred composition, approximately 18% of the volume of the preferred composition is preferably the fenugreek essential oil, approximately 18% of the volume of the preferred composition is preferably the almond essential oil, approximately 18% of the volume of the composition is preferably the mint essential oil, approximately 18% of the volume of the preferred composition is preferably the garlic essential oil, and approximately 18% of the volume of the preferred composition is preferably the castor essential oil. Even further, approximately 10% of the volume of the composition is preferably the at least one carrier oil.

In accordance with another preferred embodiment hereof, this system provides a composition for skin use, the composition preferably comprising: fenugreek essential oil, almond essential oil, at least one carrier oil, and at least one additional plant-based essential oil. Even further, the system provides such a preferred composition wherein the at least one carrier oil preferably includes fractionated coconut oil. Even further, it provides such a preferred composition wherein such at least one additional plant-base essential oil preferably includes at least one of mint essential oil, garlic essential oil, and castor essential oil. Even further, it provides such a preferred composition wherein approximately 18% of the volume of the composition is preferably the fenugreek essential oil.

The present system holds significant improvements and serves as a useful therapeutic composition for topical (skin) application. Preferably, such bio-affecting and body treating compositions should assist in providing pain relief and, yet would perform reliably and be manufactured at a modest expense. Thus, a need exists for new bio-affecting and body treating compositions that avoid the above-mentioned problems.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present disclosure, a therapeutic composition for skin use, formulated and operative according to the teachings of the present disclosure.

FIG. 1 is a chart illustrating preferred volumetric ratios of a group of constituents forming a therapeutic composition, according to a preferred embodiment of the present system.

FIG. 2 is a perspective view, illustrating the topical application of the therapeutic composition to a pain-affected body region of a user, according to the preferred embodiment of FIG. 1

FIG. 3 is a perspective view, illustrating the topical application of the therapeutic composition to the head of a user, according to the preferred embodiment of FIG. 1.

FIG. 4 is a perspective view, illustrating a kit including the therapeutic composition, according to another preferred embodiment of the present system.

The various embodiments of the present invention will hereinafter be described in conjunction with the appended drawings, wherein like designations denote like elements.

### DETAILED DESCRIPTION

As discussed above, embodiments of the present disclosure relate to bio-affecting and body treating compositions and more particularly to therapeutic compositions for skin as used to relieve pain.

Generally speaking, the therapeutic compositions of the present disclosure are formulated from 100% natural ingredients with no chemical toxicants or artificial colors. Preliminary observational studies suggest that such therapeutic compositions provide muscle pain relief, arthritis pain relief, nerve pain relief, anti-inflammatory relief (of arthrodial joint pain), and headache relief. The compositions are administered topically, generally by massaging the compositions into the skin of the affected area(s). Users have reported reductions in muscle pain and arthritis pain in as little as two minutes from the time of initial application.

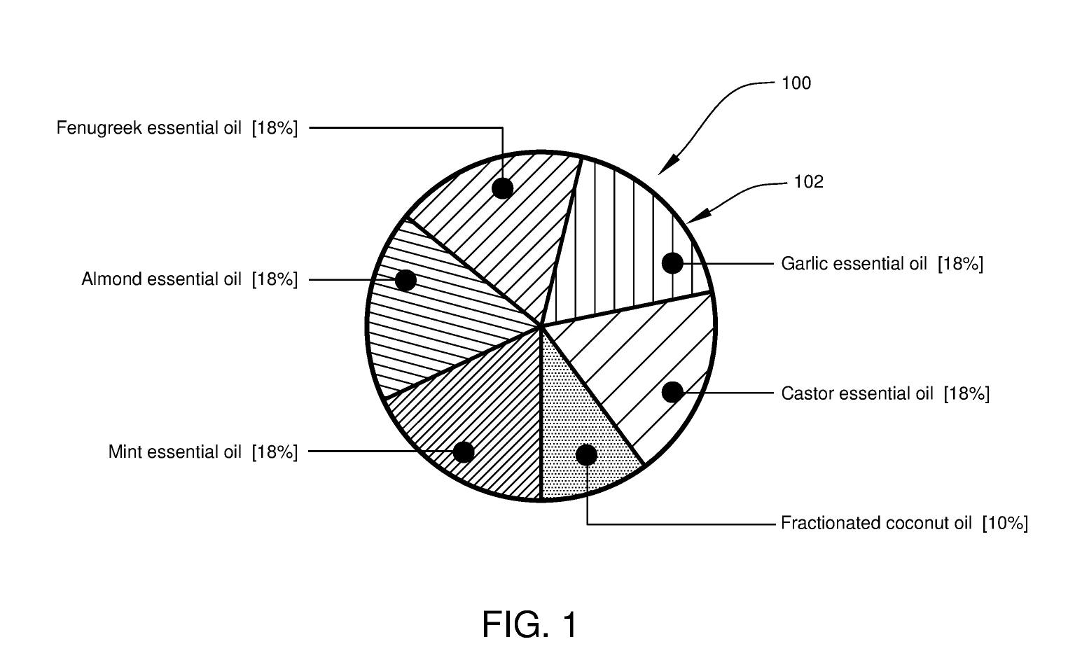

Referring now more specifically to the drawings by numerals of reference there is shown in FIGS. 1-4, various aspects of the therapeutic compositions and uses thereof for alleviating pain systems 100. In a preferred embodiment of the present system, a topically-applied therapeutic composition 102 for the treatment of pain is provided.

In specific reference to FIG. 1, there is shown a chart illustrating a preferred formulation of therapeutic composition 102 embodying the present system. Therapeutic composition 102 is preferably formulated from fenugreek essential oil, almond essential oil, mint essential oil, garlic essential oil, castor essential oil, and at least one carrier oil. Preferred carrier oils include at least one unscented oil derived from one or more natural plant sources. In the presently-disclosed embodiment, the preferred carrier oil is fractionated coconut oil.

One of the main aspects of the present system is the preferred use of all natural (non-synthetic) essential oils in the preferred formulations, which is directed to enhancing the safety and effectiveness of therapeutic composition 102. More specifically, therapeutic composition 102 is an additive composition blend composed exclusively of natural (non-synthetic) essential oils derived from natural plant sources. The term “natural essential oils”, as used in the present disclosure, refers to concentrated volatile aromatic compounds extracted from natural plant sources. In contrast, “synthetic” essential oils are laboratory-created, often from petroleum-based chemicals and similar non-natural constituents. The use of such artificial essential oils is common in the food and cosmetic industries because of their lower cost and chemical uniformity. It is noted that essential oils suitable for use in the present embodiments of the system may be commercially sourced, so long as the volatile aromatic compounds and any neutral carrier oils used in their formulation are derived from natural plant materials and are substantially free from chemicals toxicants and artificial colors, and preservatives. The use of fractionated coconut oil as the carrier oil is preferred due in part to the exceptional quality of the oil produced by the refinement process, lack of contaminants, and apparent efficacy in the formulation of the present disclosure.

FIG. 1 further illustrate preferred volumetric ratios of the preferred constituents forming therapeutic composition 102. In such a preferred composition, approximately 18% of the volume is preferably the fenugreek essential oil, approximately 18% of the volume is preferably the almond essential oil, approximately 18% of the volume is preferably the mint essential oil, approximately 18% of the volume is preferably the garlic essential oil, approximately 18% of the volume is preferably the castor essential oil, and approximately 10% of the volume is preferably the one carrier oil in the form of fractionated coconut oil.

TABLE A includes a list of preferred essential and carrier oils used in the formulation of therapeutic composition 102. In addition, TABLE A includes a listing of botanical names and extraction processes suitable for use in the disclosed embodiment.

TABLE A

| COMPONENT | BOTANICAL NAME | EXTRACTION |
|---|---|---|
| Fenugreek | Trigonella | Steam distillation from the |
| essential oil | foenum-graecum | seeds of the fenugreek plant |
| Almond | Prunus amygdalus/dulcis | Extracted from seed by cold- |
| essential oil | | press process |
| Mint | Mentha variants | Steam distillation from the |
| essential oil | | aerial portion of plant |
| Garlic | Allium sativum L. family | Maceration of the bulb |
| essential oil | Liliaceae | followed by distillation |
| Castor | Ricinus Communis | Extracted from seed by cold- |
| essential oil | | press process |
| Fractionated | Cocos nucifera | Hydrolysis/fractional |
| coconut oil | | distillation/esterification |

The unexpected analgesic properties of therapeutic composition 102 was discovered during experimental testing directed to the development of soothing aromatic skin preparations. Subjects using the claimed composition reported unexpected reductions in muscle pain and arthritis pain in anatomical structures underlying the areas of topical application, with some individuals reporting perceptible improvements in as little as two minutes from the time of initial application. Similar correlations were reported by test subjects during subsequent testing.

Although the exact mechanism of operation remains unclear, it is possible that a synergistic interaction between the complex essential oils of the claimed composition functions to enhance or magnify one or more of the analgesic properties of the constituents. Regardless of the actual mechanism, the beneficial properties of the present system appears to be unprecedented in the prior art. Those skilled in the art of the essential-oil blending will appreciate that the combination of fenugreek, almond, mint, garlic, and castor essential oils is highly unusual, which may explain the lack of prior art with regard to the claimed composition and above-described beneficial properties.

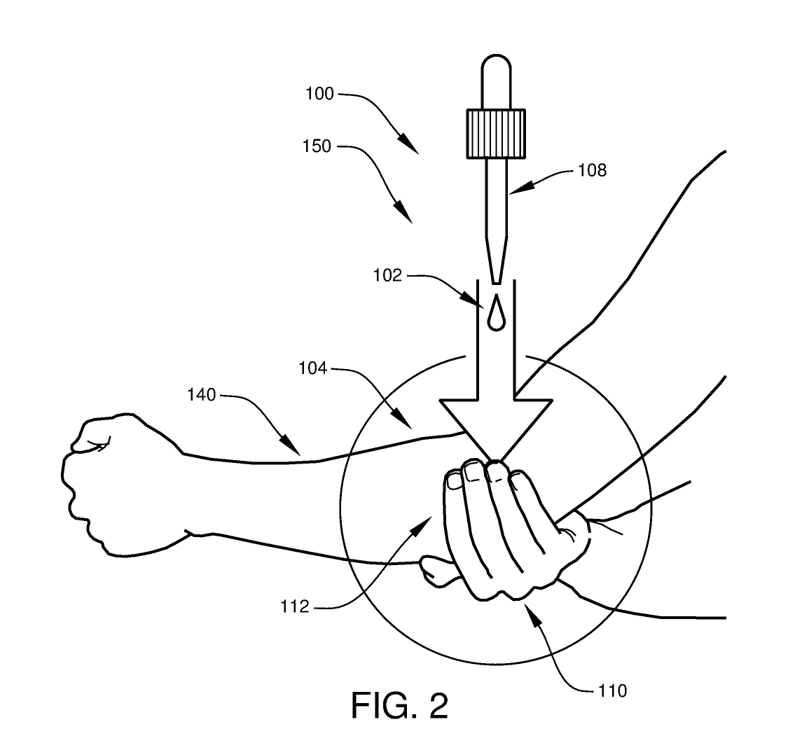

FIG. 2 is a perspective view, illustrating the topical application of therapeutic composition 102 to a pain-affected body region 104 of a user 140, according to an ‘in-use’ condition 150 of the preferred embodiment of FIG. 1. In preferred use, one or more drops of therapeutic composition 102 are applied from dispensing container 108 to the palm of the hand 110 or directly on skin 112 of the pain-affected body region 104, as shown. Therapeutic composition 102 is preferably massaged into the pain-affected body region 104 until thoroughly absorbed into skin 112. Upon reading this specification, it should be appreciated that, under appropriate circumstances, considering such issues as design preference, user preferences, marketing preferences, cost, structural requirements, available materials, technological advances, etc., other application arrangements such as, for example, utilizing topical patches infused with essential oil compositions, utilizing an aerosol applicator, utilizing a roll-on applicator, etc., may be sufficient.

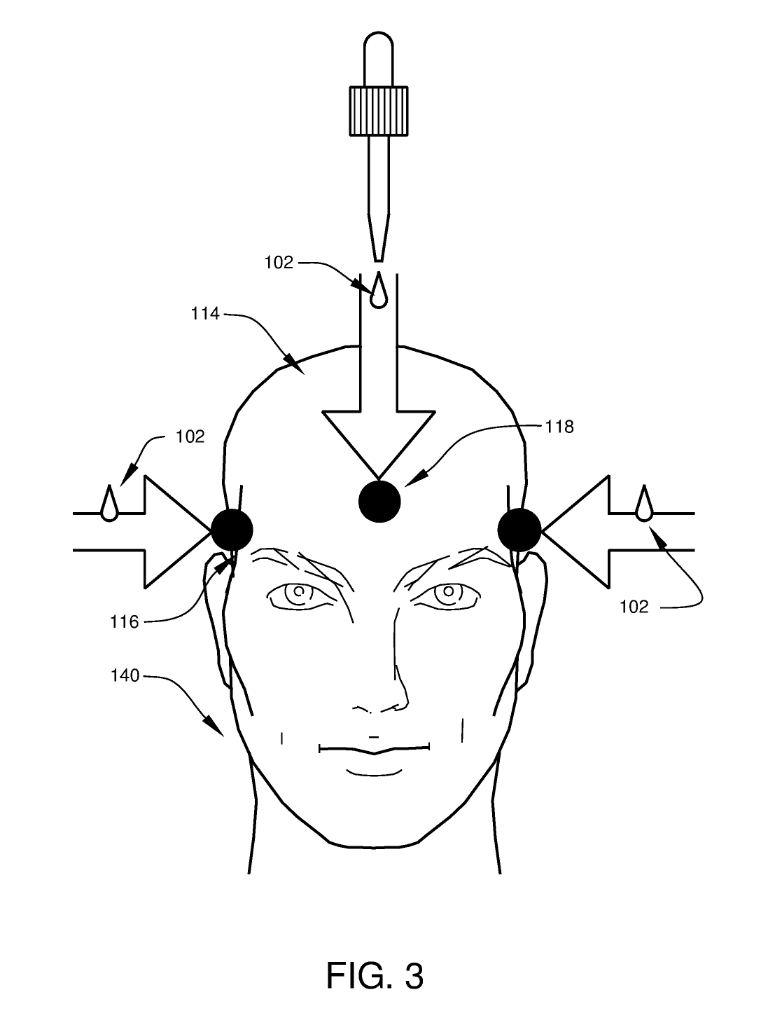

FIG. 3 is a perspective view, illustrating the topical application of therapeutic composition 102 to the head 114 of user 140, according to the preferred embodiment of FIG. 1. For headache relief, one drop of therapeutic composition 102 is applied to each temple 116 of the head 114 of user 140 and one drop of therapeutic composition 102 is applied to the top of the forehead region 118 of the head 114 of user 140, as shown. Therapeutic composition 102 is then massaged into the pain-affected body region 104 until thoroughly absorbed into the skin.

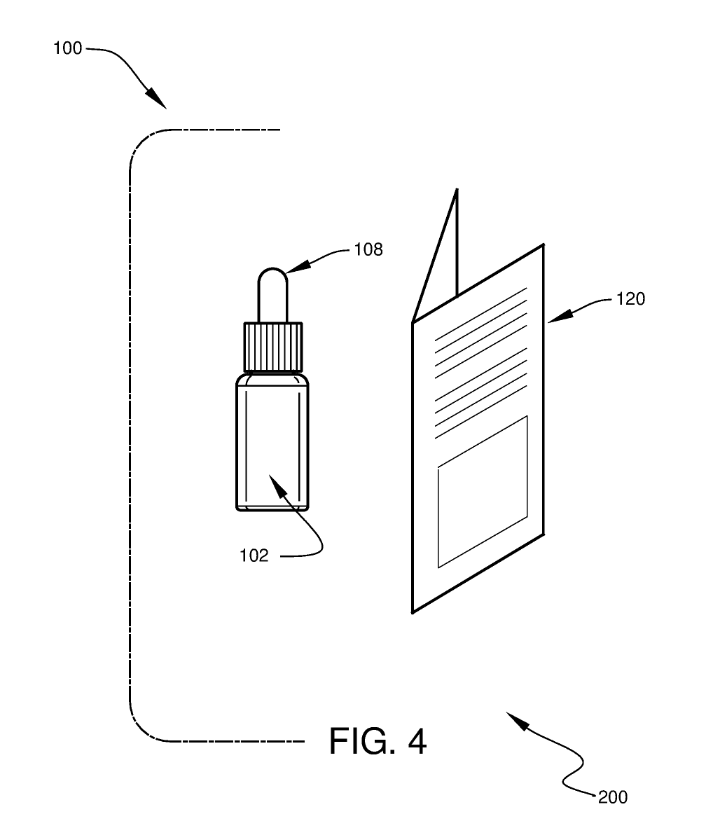

FIG. 4 is a perspective view, illustrating a kit 200 including therapeutic composition 102, according to another preferred embodiment of the present system. Therapeutic compositions and uses thereof for alleviating pain systems 100 may be sold as a kit 200 comprising the following parts: at least one dispensing container 108 configured to hold and dispense a quantity of therapeutic composition 102 and at least one set of user instructions 120. The kit has instructions such that functional relationships are detailed in relation to the composition of the embodiment (such that the system can be used, maintained, or the like in a preferred manner).

Therapeutic compositions and uses thereof for alleviating pain systems 100 may be supplied for sale in a wide variety of volumes, containers, and quantities for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized applicators, different dispenser combinations, parts may be sold separately, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A composition for skin, the composition comprising:
fenugreek essential oil;
almond essential oil;
mint essential oil;
garlic essential oil;
castor essential oil; and
at least one carrier oil.

2. The composition of claim 1, wherein said at least one carrier oil includes fractionated coconut oil.

3. The composition of claim 1, wherein approximately 18% of the volume of the composition is the fenugreek essential oil.

4. The composition of claim 1, wherein approximately 18% of the volume of the composition is the almond essential oil.

5. The composition of claim 1, wherein approximately 18% of the volume of the composition is the mint essential oil.

6. The composition of claim 1, wherein approximately 18% of the volume of the composition is the garlic essential oil.

7. The composition of claim 1, wherein approximately 18% of the volume of the composition is the castor essential oil.

8. The composition of claim 1, wherein approximately 10% of the volume of the composition is the at least one carrier oil.

9. The composition of claim 1, wherein approximately 18% of the volume of the composition is the fenugreek essential oil;
wherein approximately 18% of the volume of the composition is the almond essential oil;
wherein approximately 18% of the volume of the composition is the mint essential oil;
wherein approximately 18% of the volume of the composition is the garlic essential oil;
wherein approximately 18% of the volume of the composition is the castor essential oil; and
wherein approximately 10% of the volume of the composition is the at least one carrier oil.

10. A composition for skin use, the composition comprising:
fenugreek essential oil;
almond essential oil;
at least one carrier oil; and
at least one additional plant-based essential oil.

11. The composition of claim 10, wherein said at least one carrier oil includes fractionated coconut oil.

12. The composition of claim 11, wherein said at least one additional plant-base essential oil includes at least one of mint essential oil, garlic essential oil, and castor essential oil.

13. The composition of claim 10, wherein approximately 18% of the volume of the composition is the fenugreek essential oil.

14. The composition of claim 10 further comprising a kit including:
at least one container configured to hold a quantity of said composition for skin use; and
at least one set of user instructions.
